(19) United States
(12) Patent Application Publication
(10) Pub. No.: US 2007/0191269 A1
(43) Pub. Date: Aug. 16, 2007

(54) Medicament comprising ptx3, alone or in combination with tsg-6, for treating degenerative diseases of cartilage and bone and treating female infertility

(76) Inventors: Alberto Mantovani, Milan (IT); Paolo Carminati, Milan (IT); Barbara Bottazzi, Milan (IT)

Correspondence Address: NIXON & VANDERHYE, PC, 901 NORTH GLEBE ROAD, 11TH FLOOR, ARLINGTON, VA, 22203, US

(21) Appl. No.: 10/584,295

(22) PCT Filed: Dec. 21, 2004
(86) PCT No.: PCT/IT04/00745

§ 371 Date: Jun. 23, 2006

(30) Foreign Application Priority Data
Dec. 23, 2003 (IT) RM2003A000595

(51) Int. Cl. A61K 38/17

(52) U.S. Cl. 514/12

(57) ABSTRACT

The use of pentraxin PTX3 and of its combination with TSG-6 for the preparation of a medicament for the treatment of bone or cartilage diseases and for the treatment of female infertility is described.

The invention described herein relates to the use of long pentraxin PTX3 (PTX3) or one of its functional derivatives, alone or in combination with TSG-6 for the preparation of a medicament for the treatment of diseases of cartilage and bone and for the treatment of infertility in women.

### BACKGROUND TO THE INVENTION

PTX3 is a protein expressed in various cell types (Bottazzi, et al., J. Biol. Chem., 1997; 272: 32817-32823), particularly in mononuclear phagocytes and endothelial cells after exposure to the inflammatory cytokines Interleukin lbeta (IL-1beta) and Tumor Necrosis Factor alpha (TNF-alpha).

To date, the biological function of PTX3 has yet to be fully understood.

This protein consists of two structural domains, an N-terminal unrelated to any known molecule, and a C-terminal similar to the short pentraxins such as C-reactive protein (CRP). A substantial similarity has been found between human PTX3 (hPTX3) and animal PTX3s.

The PTX3 gene is located on mouse chromosome 3, in a region similar to the human 3q region (q24-28), in keeping with the documented location of hPTX3 in the3q 25 region. Moreover, mouse PTX3 (mPTX3) (Initrona, M., et al.: Blood, 87 (1996); 1862-1872) is very similar to hPTX3 on the basis of organisation, location and sequence (Breviario, F., et al.: J. Biol. Chem., 267: 22190, 1992).

In particular, the degree of identity between the sequences is 82% between the human gene and the mouse gene, and reaches 92% if conservative substitutions are considered.

The high degree of similarity between the sequence of hPTX3 and that of mPTX3 is a sign of the high degree of conservation of pentraxin in the course of evolution (Pepys, MB, Baltz. ML: Adv. Immunol., 34: 141, 1983).

For an overview of the pentraxins, see H. Gewurz, et al., Current Opinion in Immunology, 1995, 7: 54-64.

Previous uses of PTX3 are already known.

The international patent application WO99/32516, filed in the name of the present applicant, describes the use of long pentraxin PTX3 for the therapy of infectious, inflammatory or tumoral diseases.

WO02/38169 describes the use of long pentraxin PTX3 for the preparation of a medicament useful for the treatment of diseases associated with abnormal activation of growth factor FGF-2.

WO02/36151 describes the use of long pentraxin PTX3 for the treatment of autoimmune diseases.

WO03/011326 describes the use of long pentraxin PTX3 for the treatment of female infertility.

WO03/084561 describes the use of long pentraxin PTX3 for the preparation of a medicament for the treatment of tumoral diseases associated with abnormal activation of growth factor FGF-8.

U.S. Pat. No. 5,767,252 describes a neuronal cell growth factor belonging to the pentraxin family (see also the literature cited therein). This patent relates to the neurobiology sector.

TSG-6 is a protein inducible by inflammatory stimuli such as TNF; it is produced by different types of cells, including fibroblasts and connective tissue cells.

TSG-6 consists of two domains, a CUB domain and a LINK domain. The LINK domain of TSG-6 binds hyaluronic acid. In addition, TSG-6 binds to inter-α-trypsin inhibitor (IαI). The interaction of TSG-6 with IαI is probably important in the assembly of matrices rich in hyaluronic acid (Caroline M. Miller and Anthony J. Day, J. of Cell Science, 2003, 116(10): 1863-73).

U.S. Pat. No. 6,518,401 describes the TSG-6 protein (tumor necrosis factor stimulated gene 6 (TSG-6) protein).

U.S. Pat. No. 6,210,905 describes TSG-6 binding molecules (tumor necrosis factor stimulated gene 6 (TSG-6) binding molecules).

U.S. Pat. No. 5,846,763 describes the DNA encoding TSG-6 [DNA encoding tumor necrosis factor stimulated gene 6 (TSG-6)]

U.S. Pat. No. 5,386,013 describes TSG-6 (tumor necrosis factor-induced protein 6 (TSG-6)).

Moreover, in J., Biol., Chem., 2002, Dec. 27; 277(52): 51068-76. Epub 2002 Oct. 24, it is reported that the intravenous administration of TSG-6 reduces the concentration of a number of inflammation mediators and is endowed with anti-inflammatory activity.

In Development, 2003, May; 30(10): 2253-61 it is reported that there is distinct evidence that TSG-6 is a key catalyst in the formation of the cumulus of the extracellular matrix and is indispensable for female fertility.

In Arthritis Rheum., 2002, August 46(8): 2207-18 it is reported that the cartilage-specific constitutive expression of TSG-6 affords a chondroprotective effect but not an anti-inflammatory effect for antigen-specific arthritis. In this study, it is suggested that TSG-6 is capable of protecting the cartilage even in the presence of acute inflammation.

Many bone and cartilage diseases are associated with arthritis of various types, but not only with the latter. Schematically, two main events can be identified in arthritis: 1) inflammation, production of cytokines and swelling of the joint and: 2) degeneration of the cartilage and erosion of the bone.

The two events are commonly described as being consequential (cause and effect), but from the therapeutic point of view the idea is emerging that bone erosion and cartilage degeneration can be prevented without this inhibiting the inflammation (Glant, et al., 2002, Arthritis & Rheumatism, 46: 2207-2218). Various serine proteases are known to play a fundamental role in the events of bone and cartilage degeneration (Ronday, et al., 1996, Br. J. Rheumatol. 35. 416-23). Their activity is described as liable to modulation as a function of the degree of organisation and the composition of the extracellular matrix.

The present invention relates to a protective role of PTX3 alone or in combination with TSG-6 in degenerative diseases of bone and cartilage as a result of its cohesive effect on various components of the extracellular matrix.

The molecular cohesion function of PTX3 in combination with TSG-6, at the level of the extracellular matrix, has also proved decisive in maintaining the ovarian cumulus, a structure composed of granulosa cells and matrix surrounding the oocytes.

### DESCRIPTION OF THE INVENTION

IT has now been found that TSG-6 is a new ligand of long pentraxin PTX3, and thanks to this binding PTX3 exerts a potent protective and curative effect on diseases of cartilage and bone.

One object of the present invention is therefore the use of PTX3 or one of its functional derivatives for the preparation of a medicament for the treatment of diseases of bone and cartilage.

A further object of the present invention is the use of PTX3 or one of its functional derivatives, in combination with TSG-6, for the preparation of a medicament for the treatment of bone or cartilage diseases. A non-limiting example of said bone or cartilage diseases, are selected from the group consisting of: osteoarthritis; osteoarthrosis; degenerative diseases of the joints; collagen deficiencies; cartilage or bone diseases characterised by endochondrial ossifications: primary arthritis, including, for example, rheumatoid arthritis, juvenile arthritis, undifferentiated chronic arthritis, and polyarthritis; secondary arthritis of autoimmune origin, including, for example, systemic lupus erythematosus arthritis, psoriasic arthritis, Crohn's disease arthritis; arthritis of dysmetabolic origin, including, for example, monosodium urate arthropathy, pyrophosphate arthropathy, calcium oxalate arthropathy; infectious arthritis, arthritis due to osteoporosis, aseptic osteonecrosis, benign and malignant bone tumours.

A further object of the present invention is the use of PTX3, or one of its functional derivatives, in combination with TSG-6, for the preparation of a medicament useful for improving fertility in women needing such treatment.

A further object of the present invention is the combination containing PTX3 or one of its derivatives and TSG-6.

A further object of the present invention is the use of the combination of PTX3 or one of its derivatives and TSG-6, as a medicament. A further object of the present invention comprises pharmaceutical compositions containing as their active ingredient the combination of PTX3 or one of its derivatives and TSG-6, and at least one pharmaceutically acceptable excipient and/or diluent.

The combination according to the invention is more active than the individual components both in reducing the degeneration of cartilage and bone and in improving the cohesion of oocyte-cumulus complexes, thereby enhancing female fertility.

### DETAILED DESCRIPTION OF THE INVENTION

What is meant by “long pentraxin PTX3” is any long pentraxin PTX3, that is to say, irrespective of its natural (human or animal), recombinant or synthetic origin.

What is meant by derivative is a functional analogue of long pentraxin PTX3 bearing one or more mutations, deletions, insertions or post-transductional modifications and conserving the functional ability to bind TSG-6.

The preferred type of long pentraxin PTX3 is human long pentraxin PTX3, the sequence of which is described in WO99/32516.

What is meant by TSG-6 is the TSG-6 described in U.S. Pat. No. 6,518,401 and in the other above-mentioned US patents.

As regards the aspects relating to industrial applicability, the long pentraxin PTX3 or its derivatives and the TSG-6 will be in the form of a pharmaceutical composition in which the active ingredients are solubilised and/or vehicled by pharmaceutically acceptable excipents and/or diluents, such as sterile saline solution, carboxymethylcellulose or other excipients known to the expert in the sector.

Examples of pharmaceutical compositions usable for long pentraxin PTX3 are those described in WO 99/32516.

The compounds according to the present invention can be administered by the enteral, parenteral and vaginal routes, particularly preferred pharmaceutical forms being the slow-release implant or intra-articular injection forms.

The daily dose will depend, according to the primary care physician's judgement, on the patient's weight, age and general condition.

It should be noted that the preparation of said pharmaceutical compositions, including the slow-release forms, can be done using routine techniques and instruments well known to pharmacists and to experts in pharmaceutical technology.

1. Use of PTX3 or one of its functional derivatives for the preparation of a medicament for the treatment of bone or cartilage diseases.

2. Use of PTX3 or one of its functional derivatives, in combination with TSG-6, for the preparation of a medicament for the treatment of bone or cartilage diseases.

3. Use of PTX3, or one of its functional derivatives, in combination with TSG-6, for the preparation of a medicament useful for improving fertility in women who need it.

4. Use according to claim 1, in which what is meant by PTX3 is any long pentraxin PTX3, that is to say, irrespective of its natural (human or animal), recombinant or synthetic origin.

5. Use according to claim 1, in which what is meant by derivative is a functional analogue of long pentraxin PTX3 bearing one or more mutations, deletions, insertions, or post-transductional modifications and conserving the functional ability to bind TSG-6.

6. Use according to claim 1, in which the long pentraxin PTX3 is human long pentraxin PTX3.

7. Use according to claim 1, in which said diseases are selected from the group consisting of: osteoarthritis; osteoarthrosis; degenerative diseases of the joints; collagen deficiencies; cartilage or bone diseases characterised by endochondrial ossifications: primary arthritis, including, for example, rheumatoid arthritis, juvenile arthritis, undifferentiated chronic arthritis, and polyarthritis; secondary arthritis of autoimmune origin, including, for example, systemic lupus erythematosus arthritis, psoriasic arthritis, Crohn's disease arthritis; arthritis of dysmetabolic origin, including, for example, monosodium urate arthropathy, pyrophosphate arthropathy, calcium oxalate arthropathy; infectious arthritis, arthritis due to osteoporosis, aseptic osteonecrosis, benign and malignant bone tumours.

8. Combination comprising PTX3 or one of its derivatives and TSG-6.

9. Use of the combination according to claim 8 as a medicament.

10. Pharmaceutical composition containing as its active ingredient the combination according to claim 8 and at least one pharmaceutically acceptable excipient and/or diluent.
